SEQUENCE LISTING (3 sequences)

SEQ ID NO: 1 - protein, 13 aa, Homo sapiens
CRKAAQCDPCIPG

SEQ ID NO: 2 - RNA, 25 nt, Homo sapiens
  source 1..25: mol_type = other RNA
cttgcagttttactaaaacactgaa

SEQ ID NO: 3 - RNA, 27 nt, Homo sapiens
  source 1..27: mol_type = other RNA
ttcagtgttttagtaaaactgcaaggg

(19) United States
(12) Patent Application Publication
(10) Pub. No.: US 2024/0141345 A1
(43) Pub. Date: May 2, 2024

(54) CD70-TARGETED MICELLES ENHANCE HIF2? siRNA DELIVERY AND INHIBIT ONCOGENIC FUNCTIONS IN PATIENT-DERIVED CLEAR CELL RENAL CARCINOMA CELLS

(71) Applicant: University of Southern California, Los Angeles, CA (US)

(72) Inventors: Noah Trac, Covina, CA (US); Eun Ji Paige CHUNG YOO, Rancho Palos Verdes, CA (US)

(73) Assignee: University of Southern California, Los Angeles, CA (US)

(21) Appl. No.: 18/385,174

(22) Filed: Oct. 30, 2023

Related U.S. Application Data
(60) Provisional application No. 63/420,252, filed on Oct. 28, 2022.

(51) Int. Cl. C12N 15/113; A61P 35/00; B82Y 5/00; C07K 7/08

(52) U.S. Cl. CPC C12N 15/113; A61P 35/00; B82Y 5/00; C07K 7/08; C12N 2310/11; C12N 2310/14

(57) ABSTRACT

A drug delivery for treating renal cancer, and in particular, clear cell renal carcinoma is provides. The drug delivery system includes a plurality of nanoparticles wherein each nanoparticle includes CD70-targeting peptides conjugated thereto and anti-HIF2α siRNAs conjugated thereto. A method for treating renal cancer is also provided.

### CROSS-REFERENCE TO RELATED APPLICATIONS

This application claims the benefit of U.S. provisional application Ser. No. 63/420,252 filed Oct. 28, 2022, the disclosure of which is hereby incorporated in its entirety by reference herein.

### STATEMENT REGARDING FEDERALLY SPONSORED RESEARCH OR DEVELOPMENT

This invention was made with government support under grant no. DP2-DK121328, awarded by the NIH (National Institutes of Health)/NHLBI (National Heart, Lung, and Blood Institute). The government has certain rights in the invention.

### SEQUENCE LISTING

The XML file USC0340 of size 3,723 bytes created Oct. 29, 2023 filed herewith, is hereby incorporated by reference.

### TECHNICAL FIELD

In at least one aspect, the present invention is related to treatments for renal disease and in particular, renal carcinoma.

### BACKGROUND

Clear cell renal cell carcinoma (ccRCC) comprises 75-80% of all kidney cancers, with a five-year survival rate of 40% and a 50% risk of metastasis [1-3]. The current clinical standard for the systemic treatment of ccRCC consists of combination therapies using an immune checkpoint inhibitor (ICI), such as anti-PD1 or anti-PDL1 antibodies, plus a VEGFR tyrosine kinase inhibitor (TKI). However, adverse events (AEs) related to treatment toxicity remains common [4]. For example, in the JAVELIN Renal 101 phase lb trial including 434 patients treated with a combination of the anti-PDL1 antibody avelumab and the TKI axitinib, 71% of patients experienced a grade 3 or worse AE, resulting in dose reduction or treatment discontinuation in half of the patients [5]. Additionally, in the CLEAR phase III clinical trial in which 352 patients were treated with a combination of pembrolizumab and the TKI lenvatinib, grade 3-4 AEs were observed in 82% of patients, resulting in 68% of patients receiving dose reductions and another 13% discontinuing treatment [6]. These recent clinical trials indicate that combination ICI-TKI therapy is associated with severe toxicities, and thus, the development of efficacious alternative therapies that minimize patient AEs remains a major need.

In an effort to boost treatment efficacy and minimize toxicity, therapies targeted to specific molecular targets of ccRCC have been recently explored. For example, 70-90% of ccRCCs are characterized by loss of function of the Von Hippel-Lindau (VHL) tumor suppressor gene, which suppresses the activity of and destabilizes hypoxia-induced factors (HIFs) [7-11]. This has generated interest in the development of therapies targeting HIFs, especially HIF2α, as increased HIF2α expression is linked to upregulation of oncogenes like SLC2A1, CCND1, VEGFA, CXCR4, and CXCL12 that support cancer progression through increased glucose transport and glycolysis, cell cycle progression, angiogenesis, and cell migration [12-14]. Though the FDA approval of an orally administered HIF2α inhibitor (belzutifan) in 2021 represents a significant step forward in the treatment of ccRCC, 90% of treated patients experience anemia resulting from HIF2α suppression in non-target cells, namely hepatocytes [15-18]. Therefore, there is many opportunities for novel delivery strategies to reduce off-target toxicities in HIF2α therapies.

Nanoparticles can minimize toxicity by encapsulating therapeutic cargo in a biocompatible carrier, and the size, charge, and surface properties of the nanoparticle can be tuned to increase specific delivery to target tissues [19-23]. For example, studies by our and other groups have shown that nanoparticles less than 100 nm in size achieve the greatest kidney accumulation [24-27].

Accordingly, there is a need for improved methods for treating renal cancer.

### SUMMARY

In at least one aspect, a drug delivery system for treating renal cancer, and in particular, clear cell renal carcinoma is provided. The drug delivery system includes a plurality of nanoparticles wherein each nanoparticle includes targeting peptides and/or targeting antibodies and/or targeting aptamers that target a renal carcinoma marker conjugated thereto and a therapeutic for renal carcinoma conjugated thereto, the therapeutic for renal carcinoma selected from the group consisting of siRNAs, microRNAs, mRNA, DNA, nucleic acids, small molecule drugs, and combinations thereof

In another aspect, a drug delivery system for treating renal cancer, and in particular, clear cell renal carcinoma is provided. The drug delivery system includes a plurality of nanoparticles wherein each nanoparticle includes CD70-targeting peptides conjugated thereto and anti-HIF2a siRNAs conjugated thereto.

In another aspect, a method for treating renal cancer is also provided. The method includes steps of identifying a subject having renal cancer and administering a therapeutically effective amount of the drug delivery system set forth herein to the subject.

In another aspect, a peptide amphiphile micelles (PAMs) that are 8-20 nm in size, incorporate siRNA targeted to HIF2α, and are functionalized with a targeting ligand capable of binding to CD70, a transmembrane protein found to be uniquely expressed in ccRCCs is provided. The chosen targeting ligand is a 13-mer peptide derived from CD27, the binding partner of CD70 [29]. After successfully developing HIF2α-CD27 PAMs, we evaluated their siRNA release profile under intracellular glutathione concentrations. Then, to assess the efficacy of HIF2α-CD27 PAMs, we evaluated micelle binding to human patient tissue-derived ccRCC cells in vitro and evaluated their ability to inhibit cancer cell glucose transport, proliferation, release of angiogenic factors, and migration. Overall, the in vitro efficacy of HIF2α-CD27 PAMs in downregulating multiple oncogenic mechanisms and their clinical potential for ccRCC gene therapy are provided.

The majority of clear cell renal cell carcinomas (ccRCCs) are characterized by mutations in the Von Hippel Lindau (VHL) tumor suppressor gene, which leads to the stabilization and accumulation of the HIF2α transcription factor that upregulates oncogenes that promote glucose metabolism, cell cycle progression, angiogenesis, and cell migration. Although drugs including immune checkpoint inhibitors (ICIs), tyrosine kinase inhibitors (TKIs), and HIF2α inhibitors are FDA approved for treating ccRCC, they are associated with severe adverse effects stemming from off-target toxicity. To improve ccRCC-specific drug delivery, peptide amphiphile micelles (PAMs) were synthesized incorporating peptides targeted to the CD70 marker expressed by ccRCs and anti-HIF2α siRNA, and the ability of HIF2α-CD27 PAMs to modulate HIF2α and its downstream targets was evaluated in human ccRCC patient-derived cells. Cell cultures were derived from 8 human ccRCC tumors and the baseline mRNA expression of HIF2α and CD70, as well as the HIF2α target genes SLC2A1, CCND1, VEGFA, CXCR4, and CXCL12 were first determined. As expected, each gene was overexpressed by at least 63% of samples compared to normal kidney proximal tubule cells. Upon incubation with HIF2α-CD27 PAMs, a 50% increase in ccRCC-binding was observed upon incorporation of a CD70-targeting peptide into the PAMs, and gel shift assays demonstrated the rapid release of siRNA (>80% in 1 h) under intracellular glutathione concentrations, which contributed to ˜70% gene knockdown of HIF2α and its downstream genes. Further studies demonstrated that knockdown of the HIF2α target genes SLC2A1, CCND1, VEGFA, CXCR4, and CXCL12 led to inhibition of their oncogenic functions of glucose transport, cell proliferation, angiogenic factor release, and cell migration by 50-80%. Herein, the development of a nanotherapeutic strategy for ccRCC-specific siRNA delivery and its potential to interfere with key oncogenic pathways is presented.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements and wherein:

FIGS. 1A, 1B, 1C, 1D, and 1E. Physicochemical characterization of HIF2α-CD27 PAMs. A) Schematic of HIF2α-CD27 PAM self-assembly. B) Transmission electron micrograph of HIF2α-CD27 PAMs. C) Zeta potential of CD27 PAMs before and after HIF2α siRNA incorporation at 1 mol %. D) Gel electrophoresis assay demonstrating incorporation of siRNA into PAMs at 1 mol % and protection of siRNA from RNAse-mediated degradation after 1 h. Lanes: i) HIF2α-CD27 PAM, ii) HIF2α siRNA, iii) HIF2α-CD27 PAM incubated with RNAse-treated FBS for 1 h, iv) HIF2α siRNA incubated with RNAse-treated FBS for 1 h. E) Gel electrophoresis assay with PAMs up to 24 h after GSH treatment demonstrates rapid siRNA release following exposure to intracellular GSH levels (1 mM) and minimal siRNA release (<25%) after extracellular GSH exposure (1 μM). Scale bar=200 nm.

FIGS. 2A, 2B, 2C, 2D, 2E, 2F, and 2G. Baseline mRNA expression in patient-derived ccRCC cell cultures. Patient-derived ccRCC cells have increased A) HIF2A, B) CD70, C) SLC2A1, D) CCND1, E) VEGFA, F) CXCR4, G) CXCL12 mRNA expression compared to HK-2 kidney epithelial cells. N=8. *p<0.05, **p<0.001, ****p<0.0001.

FIGS. 3A, 3B, 3C, 3D, 3E, 3F, and 3G. In vitro binding to patient-derived ccRCC cells and to tumor tissue sections. Confocal microscopy images of patient-derived ccRCC cells that were immunostained for CD70 (green), counterstained with DAPI (blue), and incubated for 30 min with 50 1.tM FITC-labeled micelles (red). A) CD27 PAMs have increased binding to ccRCC cells in vitro compared to B) scrCD27 PAMs. C) CD27 PAM binding is reduced after pre-incubation with anti-CD70 antibodies, confirming specificity of CD27 PAMs for CD70. D) CD27 PAMs have significantly increased binding compared to scrCD27 PAMs, but not if target cells are pre-treated with anti-CD70. N=6. *p<0.05. E) ccRCC tumor tissue sections incubated for 1 h with 10 1.tM CD27 micelles have increased nanoparticle signal compared to F) scrCD27 micelles. Scale bar=50 μm. G) Representative H&E-stained ccRCC tumor tissue section. Scale bar=100 μm.

FIG. 4. mRNA expression of HIF2A (left) and CD70 (right) following 48 h treatment with HIF2α-CD27 PAMs. 48 h HIF2α-CD27 PAM treatment (500 nM siRNA) significantly reduce HIF2A and CD70 mRNA expression compared to non-targeted PAM and free siRNA controls in patient-derived ccRCC cells. N=5. *p<0.05 relative to PBS. #p<0.05 relative to HIF2α-CD27 PAM. &p<0.05 relative to HIF2α-scrCD27 PAM.

FIGS. 5A, 5B, 5C, and 5D. Gene knockdown and functional effects of HIF2α-CD27 PAM treatment. 48 h HIF2α-CD27 PAM treatment (500 nM siRNA) reduces A) SLC2A1 mRNA expression and B) glucose transport relative to non-targeted PAMs, free siRNA, and PBS in patient-derived ccRCC cells. HIF2α-CD27 PAMs also reduce C) CCND1 mRNA expression and D) ccRCC proliferation. N=4-6. *p<0.05 relative to PBS. #p<0.05 relative to HIF2α-CD27 PAM. &p<0.05 relative to HIF2α-scrCD27 PAM.

FIGS. 6A and 6B. 48 h HIF2α-CD27 PAM treatment (500 nM siRNA) reduces A) VEGFA mRNA expression in patient-derived ccRCC. B) HUVECs cultured with HIF2α-CD27 PAM-conditioned culture medium have reduced proliferation relative to PBS-conditioned culture medium. N=5. *p<0.05 relative to PBS. #p<0.05 relative to HIF2α-CD27 PAM. &p<0.05 relative to HIF2α-scrCD27 PAM.

FIGS. 7A and 7B. Effect of HIF2α-CD27 PAMs on ccRCC migration. A) 48 h HIF2α-CD27 PAM treatment (500 nM siRNA) slows wound closure of ccRCC cells over 24 h compared to PBS and free siRNA treatment. B) 48 h HIF2α-CD27 PAM treatment (500 nM) reduces mRNA expression of CXCR4 (left) and CXCL12 (right). N=4 or 5. *p<0.05 relative to PBS. #p<0.05 relative to HIF2α-CD27 PAM.

FIG. 8. MALO/mass spectrum of CO27 PA. Expected m/z=4343.

FIG. 9. MALO/mass spectrum of scrCO27 PA. Expected m/z=4343.

FIG. 10. Agarose gel after siRNA release study.

FIG. 11. In vitro PAM binding to HK-2 cells.

FIG. 12. MTS assay of ccRCC cells 72h after transfection.

FIG. 13. Representative image analysis slides for wound healing assay, timepoints 0 h and 24 h.

### DETAILED DESCRIPTION

Reference will now be made in detail to presently preferred compositions, embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: all R groups (e.g. Rⁱ where i is an integer) include hydrogen, alkyl, lower alkyl, C₁₋₆ alkyl, C₆₋₁₀ aryl, C₆₋₁₀ heteroaryl, —NO₂, —NH₂, —N(R′R″), —N(R′R″R′″)⁺L⁻, Cl, F, Br, —CF₃, —CCl₃, —CN, —SO₃H, —PO₃H₂, —COOH, —CO₂R′, —COR′, —CHO, —OH, —OR′, —O⁻M⁺, —SO₃⁻M⁺, —PO₃⁻M⁺, —COO⁻M⁺, —CF₂H, —CF₂R′, —CFH₂, and —CFR′R″ where R′, R″ and R″′ are C₁₋₁₀ alkyl or C₆₋₁₈ aryl groups; single letters (e.g., “n” or “o”) are 1, 2, 3, 4, or 5; in the compounds disclosed herein including compounds described by formula or by name, a CH bond can be substituted with alkyl, lower alkyl, C₁₋₆ alkyl, C₆₋₁₀ aryl, C₆₋₁₀ heteroaryl, —NO₂, —NH₂, —N(R′R″), —N(R′R″R′″)⁺L⁻, Cl, F, Br, —CF₃, —CCl₃, —CN, —SO₃H, —PO₃H₂, —COOH, —CO₂R′, —COR′, —CHO, —OH, —OR′, —O⁻M⁺, —SO₃⁻M⁺, —PO₃⁻M⁺, —COO⁻M⁺, —CF₂H, —CF₂R′, —CFH₂, and —CFR′R″ where R′, R″ and R′″ are C₁₋₁₀ alkyl or C₆₋₁₈ aryl groups; percent, “parts of ” and ratio values are by weight; the term “polymer” includes “oligomer,” “copolymer,” “terpolymer,” and the like; molecular weights provided for any polymers refers to weight average molecular weight unless otherwise indicated; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits. In the specific examples set forth herein, concentrations, temperature, and reaction conditions (e.g. pressure, pH, etc.) can be practiced with plus or minus 50 percent of the values indicated rounded to three significant figures. In a refinement, concentrations, temperature, and reaction conditions (e.g., pressure, pH, etc.) can be practiced with plus or minus 30 percent of the values indicated rounded to three significant figures of the value provided in the examples. In another refinement, concentrations, temperature, and reaction conditions (e.g., pH, etc.) can be practiced with plus or minus 10 percent of the values indicated rounded to three significant figures of the value provided in the examples.

In the examples set forth herein, concentrations, temperature, and reaction conditions (e.g., pressure, pH, flow rates, etc.) can be practiced with plus or minus 50 percent of the values indicated rounded to or truncated to two significant figures of the value provided in the examples. In a refinement, concentrations, temperature, and reaction conditions (e.g., pressure, pH, flow rates, etc.) can be practiced with plus or minus 30 percent of the values indicated rounded to or truncated to two significant figures of the value provided in the examples. In another refinement, concentrations, temperature, and reaction conditions (e.g., pressure, pH, flow rates, etc.) can be practiced with plus or minus 10 percent of the values indicated rounded to or truncated to two significant figures of the value provided in the examples.

To determine the “percent identity” (i.e., percent sequence identity) of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a refinement, the sequences are aligned for maximum correspondence over a specified comparison window, as measured by sequence comparison algorithms or by visual inspection. In a refinement, the length of a first sequence aligned for comparison purposes is at least 80% of the length of a second sequence, and in some embodiments is at least 90%, 95%, or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5. In this regard, the following oligonucleotide alignment algorithms may be used: BLAST (GenBank URL: www.ncbi.nlm.nih.gov/cgi-bin/BLAST/, using default parameters: Program: BLASTN; Database: nr; Expect 10; filter: default; Alignment: pairwise; Query genetic Codes: Standard(1)), BLAST2 (EMBL URL: http://www.embl-heidelberg.de/Services/index.html using default parameters: Matrix BLOSUM62; Filter: default, echofilter: on, Expect:10, cutoff: default; Strand: both; Descriptions: 50, Alignments: 50), or FASTA, search, using default parameters. When sequences differ in conservative substitutions, the percent identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity.

Throughout this application, where publications are referenced, the disclosures of these publications in their entireties are hereby incorporated by reference into this application to more fully describe the state of the art to which this invention pertains.

In an embodiment, a drug delivery system for treating renal cancer, and in particular, clear cell renal carcinoma is provided. The drug delivery system includes a plurality of nanoparticles wherein each nanoparticle includes one or more targeting moieties that target a renal carcinoma marker conjugated thereto and a therapeutic (agent) for renal carcinoma conjugated thereto. In a refinement, the one or more targeting moieties include a component selected from the group consisting of targeting peptides, targeting antibodies, targeting aptamers, and combinations thereof. In a refinement, the therapeutic for renal carcinoma selected from the group consisting of siRNAs, microRNAs, mRNA, DNA, nucleic acids, small molecule drugs, and combinations thereof

In a variation, the drug delivery system includes a plurality of nanoparticles wherein each nanoparticle includes a one or more targeting peptides that target a renal carcinoma marker conjugated thereto and a therapeutic (agent) for renal carcinoma conjugated thereto.

In a variation, the drug delivery system includes a plurality of nanoparticles wherein each nanoparticle includes a one or more targeting antibodies that target a renal carcinoma marker conjugated thereto and a therapeutic (agent) for renal carcinoma conjugated thereto.

In a variation, the drug delivery system includes a plurality of nanoparticles wherein each nanoparticle includes a one or more targeting aptamers that target a renal carcinoma marker conjugated thereto and a therapeutic (agent) for renal carcinoma conjugated thereto.

In a variation, a drug delivery system for treating kidney cancer, and in particular, clear cell renal cancer is provided. The drug delivery system includes a plurality of nanoparticles wherein each nanoparticle includes one or more CD70-targeting moieties conjugated thereto. In a refinement, the one or more CD70-targeting moieties include a component selected from the group consisting of CD70-targeting peptides, CD70-targeting antibodies, CD70-targeting aptamers and combinations thereof In a refinement, anti-HIF2α siRNAs are also conjugated to each nanoparticle. In a refinement, the plurality of nanoparticles includes nanoparticles selected from the group consisting of micelles, liposomes, and combinations thereof

In a variation, the CD70-targeting peptides conjugated is CD27-derived peptides (CRKAAQCDPCIPG) (SEQ ID NO:1) or a peptide thereof with 1 to 7 conservative substitutions or additions. In another refinement, the CD70-targeting peptides conjugated includes a fragment of CD27 (CRKAAQCDPCIPG) (SEQ ID NO:1) having 5 to 12 amino acids or peptide thereof with 1 to 7 conservative substitutions or additions.

In a variation, a drug delivery system for treating kidney cancer, and in particular, clear cell renal cancer is provided. The drug delivery system includes a plurality of nanoparticles wherein each nanoparticle includes CD70-targeting antibodies, and in particular, CD70-targeting monoclonal antibodies conjugated thereto and optionally, anti-HIF2α siRNAs conjugated thereto. In a refinement, the plurality of nanoparticles includes nanoparticles selected from the group consisting of micelles, liposomes, and combinations thereof. An example of a CD70-targeting monoclonal antibody is ARGX-110, a defucosylated IgG1 monoclonal antibody (mAb) which is described in Silence K, Dreier T, Moshir M. Uirichts P, Gabriels S M, Saunders M. Wajant H, Brouckaert P, Huyghe L, Van Hauwermeiren T, Thibault A ; De Haard H.1. ARGX-110; a highly potent antibody targeting CD70, eliminates tumors via both enhanced ADCC and immune checkpoint blockade. MAbs, 2014 March-April;6(2):523-32. doi: 10.4161/mabs.27398. Epub 2013 Dec. 6. PIN/HD: 24492296; PMCID: PMC3984340; the entire disclosure of which is hereby incorporated by reference; the entire disclosure of which is hereby incorporated by reference in its entirety. Another example of a CD70-targeting monoclonal antibody is SGN-CD70A described in https://www.adcreview.com/drugmap/sgn-cd70-a-sup erseding- sgn-75/ and in https://doi.org/10.1002/cncr.31912; the entire disclosures of which is hereby incorporated by reference in its entirety.

In a variation, a drug delivery system for treating kidney cancer, and in particular, clear cell renal cancer is provided. The drug delivery system includes a plurality of nanoparticles wherein each nanoparticle includes CD70-targeting aptamer conjugated thereto and optionally, anti-HIF2a siRNAs conjugated thereto. An example of a CD70-targeting aptamer is Aptamer 928 described in Payam Bayat, Seyed Mohammad Taghdisi, Houshang Rafatpanah, Khalil Abnous, Mohammad Ramezani, In vitro selection of CD70 binding aptamer and its application in a biosensor design for sensitive detection of SKOV-3 ovarian cells, Talanta, Volume 194, 2019, Pages 399-405, ISSN 0039-9140,B https://doi.org/10.1016/j.talanta.2018.10.063. (https://www.sciencedirect.com/science/article/pii/S0039914018311032); the entire disclosure of which is hereby incorporated by reference in its entirety.

In a variation, the anti-HIF2α siRNAs include a polynucleotide having sequence 5′ -CUUGCAGUUUUACUAAAACACUGAA-3′ (SEQ ID NO: 2) and/or an antisense sequence: 5′ -UUCAGUGUUUUAGUAAAACUGCAAGGG-3′ (SEQ ID NO: 3) or sequences having a sequence identity greater than 80 percent identity thereof. In a refinement, the anti-HIF2α siRNAs include a fragment of a polynucleotide having sequence 5′ -CUUGCAGUUUUACUAAAACACUGAA-3′ (SEQ ID NO: 2) and/or an antisense sequence: 5′-UUCAGUGUUUUAGUAAAACUGCAAGGG-3′ (SEQ ID NO: 3) having 5 to 30 nucleotides or sequences having a sequence identity greater than 80 percent identity thereof.

Advantageously, the CD70-targeting peptides and the anti-HIF2α siRNAs are independently connected to the nanoparticles by reaction with a functional group. For example, the functional group that can be used for linking includes amines, carboxylic acids, NHS esters, acid anhydrides, or unsaturated imides (e.g., maleimide). In another refinement, the CD70-targeting peptides and the anti-HIF2α siRNAs are independently absorbed electrostatically to the nanoparticles.

As set forth above, the plurality of nanoparticles can include a plurality of micelles. In this regard, each micelle can include a plurality of targeting peptide-conjugated amphiphiles, a plurality of therapeutic agent-conjugated amphiphiles, and an optional plurality of non-targeted amphiphiles. In a refinement, the plurality of targeting peptide-conjugated amphiphiles includes amphiphiles having a first phospholipid conjugated to the CD70-targeting peptides with a first linking group, the plurality of therapeutic agent-conjugated amphiphiles includes amphiphiles having a second phospholipid conjugated to the anti-HIF2α siRNAs with a second linking group, and the optional plurality of non-targeted amphiphiles includes amphiphiles having a third phospholipid conjugated to a capping moiety with a third linking group. In a further refinement, the first linking group, the second linking group, and the third linking group are each independently a polyethylene glycol having a weight average molecular weight from about 500 to 10000 Daltons. In a further refinement, the first phospholipid, the second phospholipid, and the third phospholipid are each independently selected from the group consisting of phosphatidic acids, phosphatidyl inositols, phosphatidyl cholines, phosphatidyl ethanolamines, phosphatidyl serines, phosphatidyl glycerols, and any combinations thereof. In some examples, the first phospholipid, the second phospholipid, and the third phospholipid are each independently selected from the group consisting of phosphatidylglycerol, lecithin, sphingomyelin, phosphatidylserine, phosphatidic acid, N-(2,3-di(9-(Z)-octadecenyloxy))-prop-1-yl-N,N,N-trimethylammonium chloride, phosphatidylethanolamine, lysol ecithin, lysophosphatidylethanolamine, phosphatidylinositol, cephalin, cardiolipin, cerebrosides, dicetylphosphate, dioleoylphosphatidylcholine, dipalmitoylphosphatidylcholine, dipalmitoylphosphatidylglycerol, dioleoylphosphatidylglycerol, palmitoyl-oleoyl-phosphatidylcholine, di-stearoyl-phosphatidylcholine, stearoyl-palmitoyl-phosphatidylcholine, di-palmitoyl-phosphatidylethanolamine, di-stearoyl-phosphatidylethanolamine, di -myrstoyl-phosphatidylserine, di-oleyl-phosphatidylcholine, dimyristoyl phosphatidyl choline (DMPC), dioleoylphosphatidylethanolamine, palmitoyloleoylphosphatidylcholine, di stearoylphosphatidylcholine, dioleoylphosphatidylcholine, dipalmitoylphosphatidylcholine, dioleoylphosphatidylglycerol, dipalmitoylphosphatidylglycerol, -phosphatidylethanolamine, dioleoyl-phosphatidylethanolamine 4-(N-maleimidomethyl)-cyclohexane-1-carboxylate (DOPE-mal), 1-stearoyl-2-oleoyl phosphatidylcholine, 1,2-distearoyl-sn-glycerol-3-phosphoethanolamine, and combinations thereof.

In another variation, the drug delivery system further includes a pharmaceutically acceptable carrier.

In another variation, at least a subset of the micelles further includes a drug incorporated into the core of the micelle or conjugated to the hydrophobic tail of the amphiphiles.

In another variation, at least a subset of the micelles include a component selected from the group consisting of small molecule therapeutics, chemotherapeutic agents, monoclonal antibodies, therapeutic cytokines, and tyrosine kinase inhibitors which are currently used for treating renal carcinoma incorporated therein.

In another embodiment, a method for treating renal cancer in a subject. The method includes steps of identifying a subject having renal cancer and administering a therapeutically effective amount of the drug delivery system set forth above. In a refinement, a small molecule therapeutic is incorporated into the nanoparticles. In another refinement, the drug delivery system is administered intravenously, subcutaneously, orally, by intraperitoneal injection, and transdermally, and the like.

Additional details of the drug delivery system are found in Trac N, Oh H S, Jones L I, Caliliw R, Ohtake S, Shuch B, Chung E J. CD70-Targeted Micelles Enhance HIF2α siRNA Delivery and Inhibit Oncogenic Functions in Patient-Derived Clear Cell Renal Carcinoma Cells. Molecules. 2022 Dec. 2;27(23):8457. doi: 10.3390/molecules27238457. PMID: 36500549; PMCID: PMC9738223 and its associated supplemental material; the entire disclosure of which is incorporated by reference.

The following examples illustrate the various embodiments of the present invention. Those skilled in the art will recognize many variations that are within the spirit of the present invention and scope of the claims.

### 2. RESULTS AND DISCUSSION

### 2.1 Synthesis of HIF2α-CD27 PAMs and Characterization of siRNA Loading and Release

Based on our earlier studies developing nanoparticles that accumulate in the kidneys in vivo, peptide amphiphile micelles (PAMs) that are typically less than 20 nm in diameter and are able

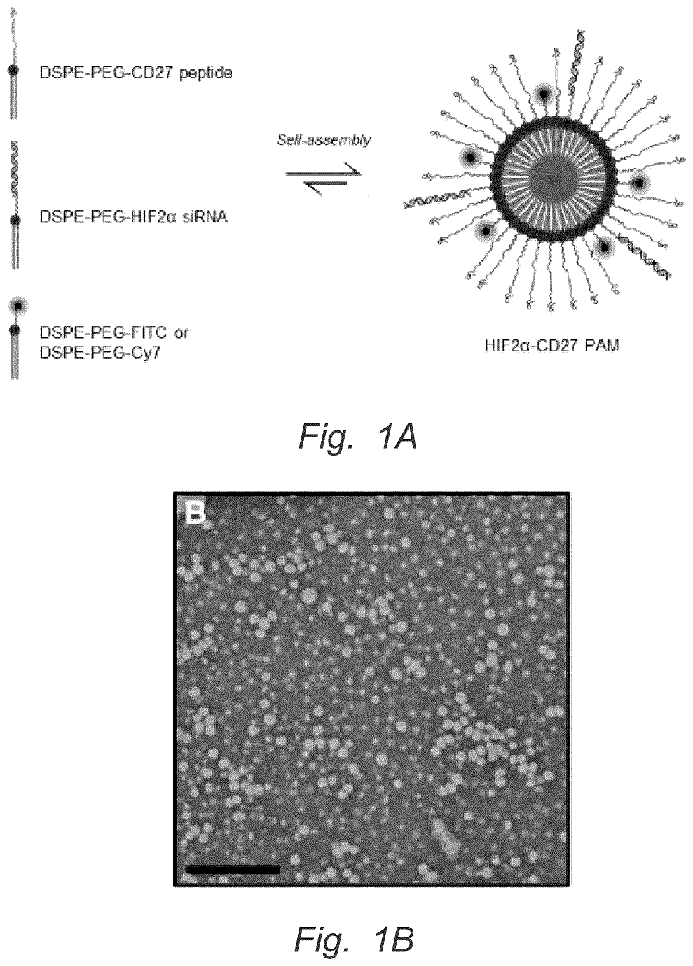

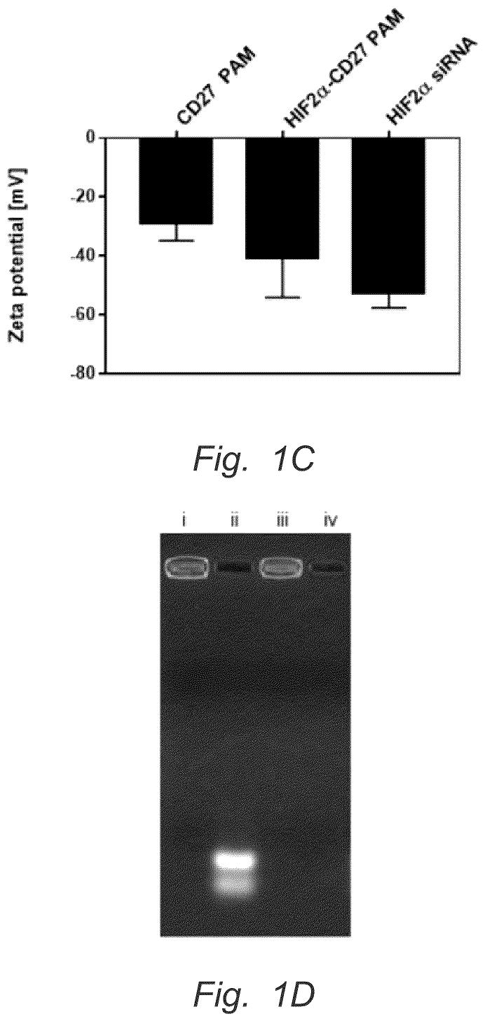

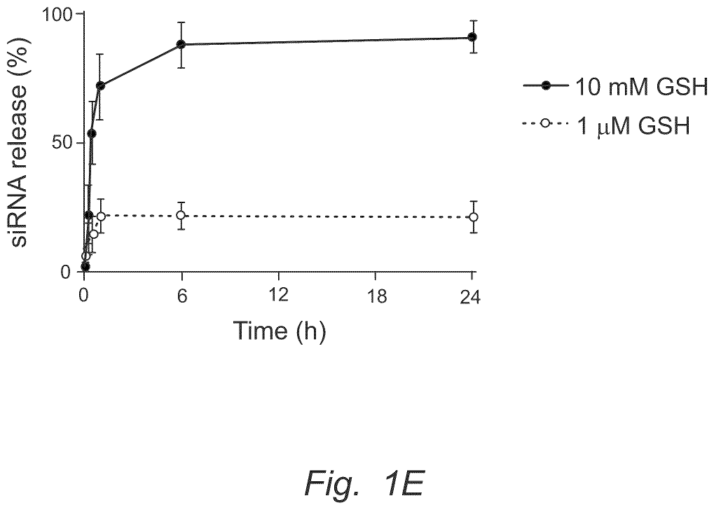

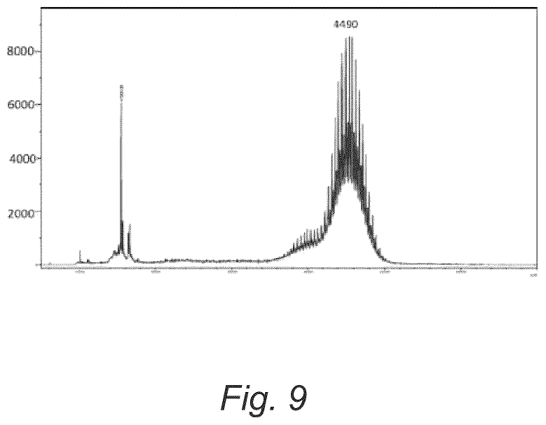

to penetrate the glomerular filtration barrier was developed for ccRCC [30]. To enhance ccRCC specificity, we incorporated a peptide targeted to the CD70 transmembrane protein expressed by ccRCCs (CD27 peptide). CD27 peptides or thiolated HIF2α siRNAs were conjugated to DSPE-PEG2000-maleimide and self-assembled into HIF2α-CD27 PAMs under aqueous conditions at a 99:1 peptide: siRNA ratio (FIGS. 1A & 8) [31-33]. Non-targeting PAMs were synthesized using scrambled CD27 peptides (FIG. 9). Transmission electron microscopy (TEM) images confirmed micelles are uniform and spherical in morphology (FIG. 1B), and dynamic light scattering (DLS) measurements showed the HIF2α-CD27 PAMs to be 13.8±0.4 nm in diameter (Table 1). Using zeta potential measurements, the surfaces of the CD27 PAMs became more negatively charged upon incorporation of the anionic siRNA as expected, from −28.8±6.0 to −40.5 ±13.6 (FIG. 1C) [34, 35].

TABLE 1
Nanoparticle Size

| Sample | Size (nm) |
|---|---|
| HIF2α-CD27 PAM | 13.8 ± 0.4 |
| CO27 PAM | 14.7 ± 0.5 |
| HIF2a siRNA | N/A |

To evaluate HIF2α siRNA incorporation into PAMs, a gel shift assay was performed to compare HIF2α-CD27 PAM (500 ng siRNA, 13.3 μg PAM) and free HIF2α siRNA (500 ng siRNA) migration [36, 37]. As shown in FIG. 1D, following 90 min of electrophoresis at 50V, >95% of HIF2α-CD27 PAMs (lane i) remained in the well, demonstrating that the HIF2α siRNA was successfully incorporated into the micelle. In contrast, the free HIF2α siRNA (lane ii) readily migrated down the gel. In addition, incorporation of the siRNA into micelles was found to protect the nucleic acid cargo from degradation, as approximately 90% of HIF2α-CD27 PAMs incubated with RNase-treated FBS for 1 h were retained in the well (lane iii), while unconjugated siRNA incubated with RNase-treated FBS for lh showed no signal in the gel (lane iv), indicating that the siRNA had been degraded into smaller oligonucleotides and migrated off the gel [38, 39].

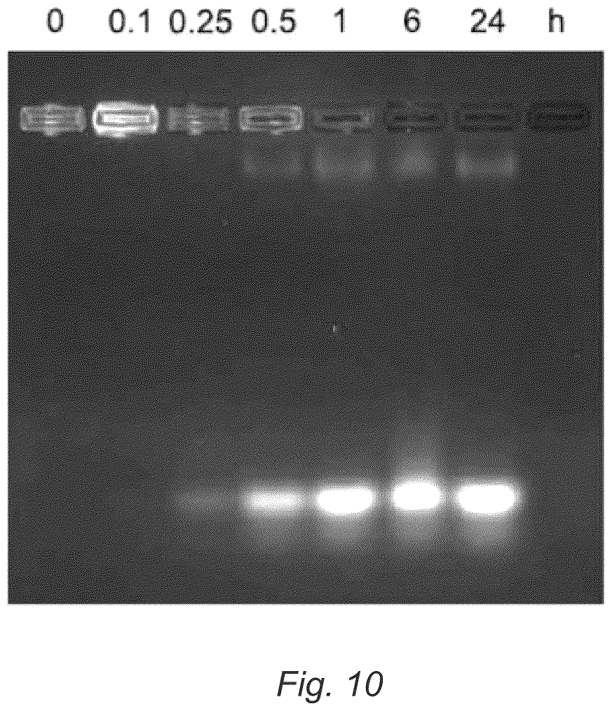

Upon internalization into the cell, HIF2α-CD27 PAMs experience intracellular levels of glutathione (GSH) up to 10 mM, which can be several orders of magnitude higher than extracellular GSH [40-44]. To characterize the release of therapeutic siRNA from the PAMs under intracellular conditions, additional gel electrophoresis assays were performed following PAM incubation with 10 mM GSH for up to 24 h [45]. siRNA release was calculated by comparing the band intensity in the well (non-released siRNA) to the band intensity further down the gel (released siRNA) (FIG. 10). As found in FIG. 1E, >80% of the siRNA was released after lh incubation with intracellular levels of GSH, indicating that HIF2α-CD27 PAMs are capable of rapidly releasing their payload after internalization into target cells. In contrast, HIF2α-CD27 PAMs incubated at extracellular concentrations of GSH (1 μM) released <25% of siRNA cargo after 24 h.

### 2.2 HIF2α and Downstream Genes are Upregulated in Patient-Derived ccRCC Cells

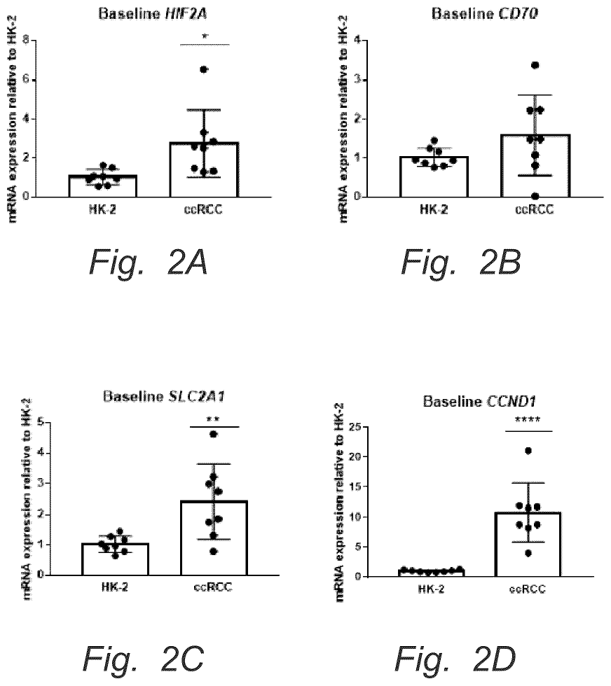

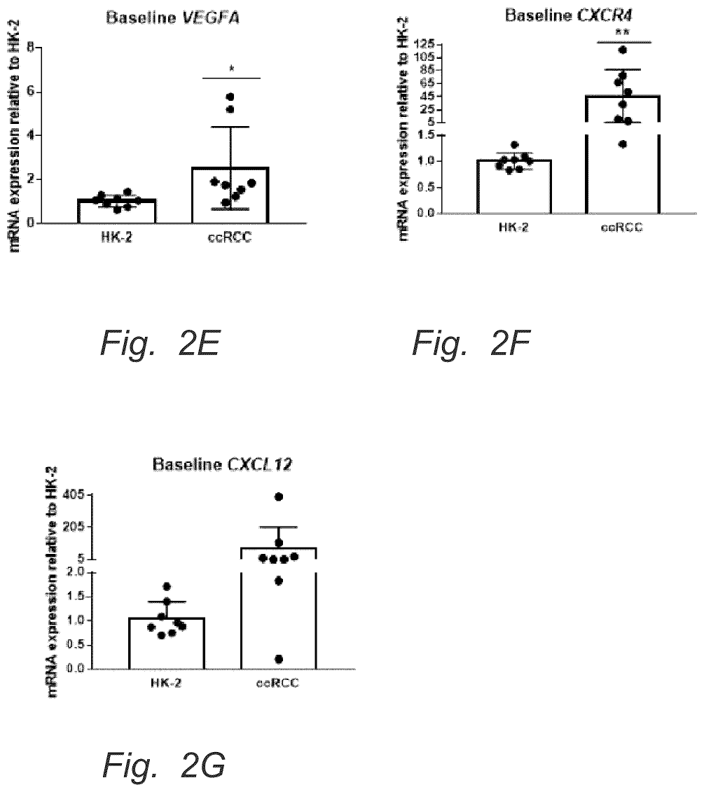

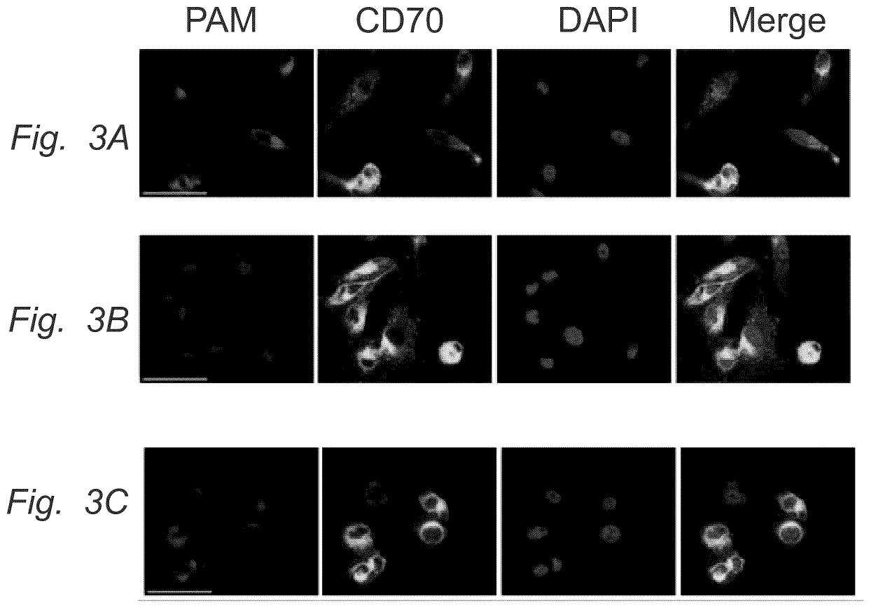

To determine if HIF2α siRNA delivery represents a viable therapeutic strategy for ccRCC patients, baseline mRNA expression of HIF2A, CD70, and cancer-supporting downstream targets regulated by HIF2α such as SLC2A1, CCND1, VEGFA, CXCR4, and CXCL12 were evaluated in HK-2 renal proximal tubule cells and eight patient-derived ccRCC cell cultures (FIGS. 2A-G) [46-51]. Patient characteristics for each of these cultures, including tumor grade, size, and stage are listed in Table 2 [52-54].

TABLE 2
ccRCC patient characteristics.

| Sample | Grade | Tumor Size (cm) | Stage |
|---|---|---|---|
| 1 | 3 | 2.5 | I |
| 2 | 2 | 6.3 | III |
| 3 | 3 | 4.7 | III |
| 4 | 3 | 11 | III |
| 5 | 2 | 3.6 | NR |
| 6 | 2 | 4.1 | I |
| 7 | 3 | 15.6 | III |
| 8 | 4 | 13.5 | III |

NR = not reported.

As found in FIG. 2, in general, the ccRCC samples had significantly higher baseline mRNA expression of HIF2A, SLC2A1, CCND1, VEGFA, and CXCR4 compared to the HK-2 kidney epithelial cell line (p<0.05). Although overall mRNA expression of CD70 and CXCL12 in the ccRCC samples was greater than that of the control HK-2 cells, the differences was not statistically significant and is attributed to the high variation in gene expression across the ccRCC samples. Analyzing the individual samples, 5 of the 8 (63%) patient-derived samples had significantly higher CD70 mRNA expression compared to the HK-2 control cells. Additionally, 7 of the 8 (88%) samples had increased CXCL12 mRNA expression (Table 3). Of the individual ccRCC samples, only Sample 5 had significantly higher mRNA expression for all the target genes compared to the HK-2 control cells

(Table 4). Because of this, we hypothesized that Sample 5 would be most susceptible to CD70-targeted HIF2α siRNA therapy and continued with this sample for subsequent in vitro studies.

TABLE 3
Patient-derived ccRCC cells with higher HIF2a-related
gene expression compared to HK-2 cells (p < 0.05).

| Gene | N (%) |
|---|---|
| HIF2A | 5/8 (63%) |
| CD70 | 5/8 (63%) |
| SLC2A1 | 5/8 (63%) |
| CCND1 | 8/8 (100%) |
| VEGFA | 7/8 (88%) |
| CXCR4 | 7/8 (88%) |
| CXCL 12 | 7/8 (88%) |

TABLE 4
mRNA expression of individual ccRCC samples, normalized to HK-2 cells.

| | Sample | | | | | | | |
|---|---|---|---|---|---|---|---|---|
| Gene | 1 | 2 | 3 | 4 | 5 | 6 | 7 | 8 |
| HIF2A | 6.54* | 3.31* | 1.28 | 1.47 | 2.84* | 1.32 | 2.61* | 2.52* |
| CD70 | 0.81 | 1.08 | 2.23* | 2.22* | 3.38* | 1.48* | 0.02* | 1.48* |
| SLC2A1 | 1.32 | 4.63* | 1.86* | 1.75* | 3.22* | 2.75* | 0.79 | 2.99* |
| CCND1 | 8.83* | 8.82* | 11.75* | 11.58* | 8.27* | 4.04* | 21.09* | 12.00* |
| VEGFA | 1.91* | 5.20* | 0.95 | 1.75* | 5.79* | 1.86* | 1.23* | 1.55* |
| CXCR4 | 116.90* | 66.95* | 33.25* | 7.40* | 10.45* | 52.47* | 1.33 | 77.82* |
| CXCL12 | 393.77* | 0.21* | 1.83* | 11.59* | 4.06* | 20.73* | 109.59* | 4.38* |

N = 3.
*p < 0.05 vs. HK-2 cells.

### 2.3 CD70-Targeting Micelles Bind to Patient-Derived ccRCC Cells in Vitro

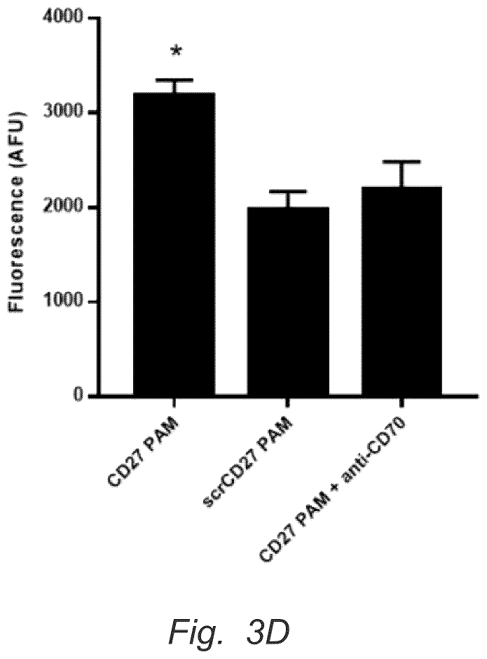

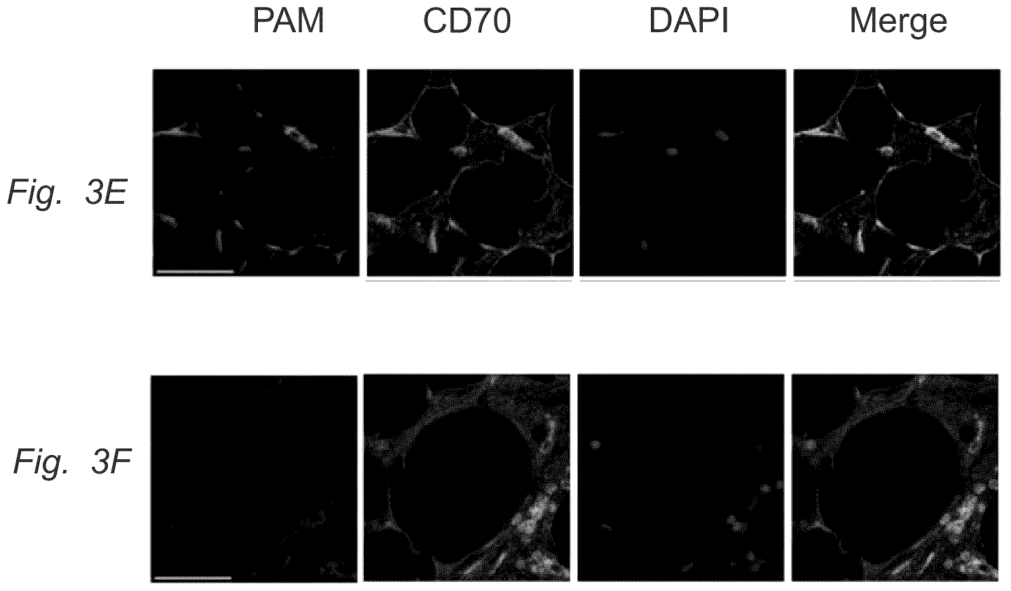

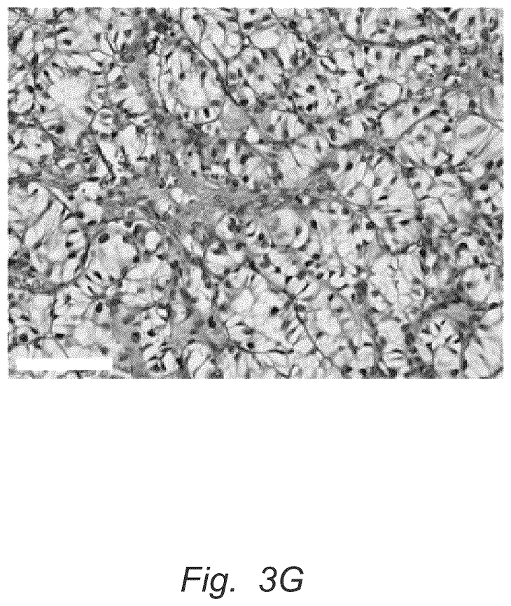

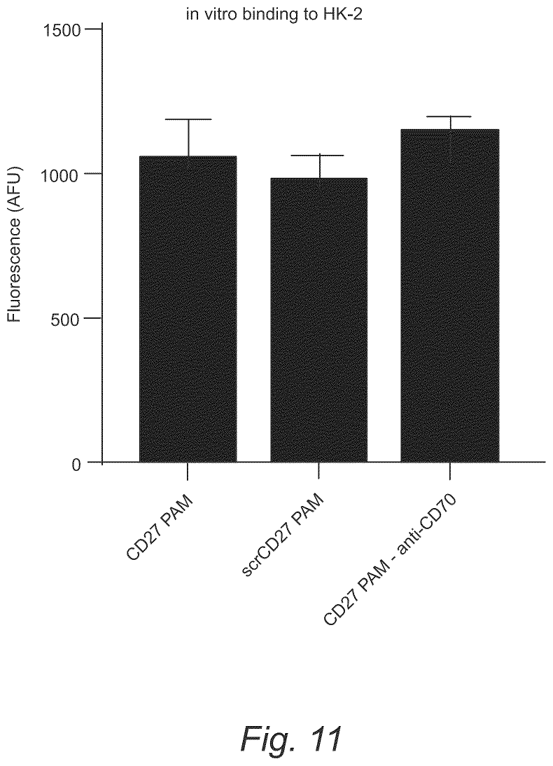

To evaluate the specificity of HIF2α-CD27 PAMs for binding to CD70+ ccRCC cells, we incubated FITC-labelled CD27 PAMs with patient-derived ccRCC cells in vitro for 30 min (FIG. 3A). Immunofluorescent antibody staining confirmed CD70 expression and that CD27 PAMs colocalized with CD70 (Pearson's colocalization coefficient R=0.54). In contrast, minimal binding was observed in cells treated with scrCD27 PAMs (FIG. 3B). To further confirm the specificity of HIF2α-CD27 PAMs for binding to CD70+ ccRCC cells, the binding of CD27 PAMs was also assessed following a 30 min incubation of the ccRCC cells with anti-CD70 polyclonal antibodies (FIG. 3C). As expected, blocking CD70 prior to PAM treatment significantly decreased PAM binding to levels comparable to the scrCD70 control. To quantify these results, we repeated these experiments with cells grown in microtiter plates and measured the fluorescence signal and found that, in agreement with the qualitative microscopy images, CD27 PAMs had significantly more binding than scrCD27 PAMs, and that this increased binding was not observed if the cells were pre-treated with anti-CD70 (FIG. 3D). The specificity of CD27 PAMs for CD70 is also apparent through our binding studies with the HK-2 cell line, which has been reported to have minimal CD70 expression [55]. When incubated with HK-2 cells for 30 min, no differences in micelle binding were observed between CD27 PAMs, scrCD27 PAMs, or CD27 PAMs after anti-CD70 incubation (FIG. 11).

In addition to patient-derived cells, binding studies were also performed ex vivo using formalin-fixed paraffin-embedded (FFPE) ccRCC tumor tissue sections by treating sections with fluorescently-labeled 10 μM PAMs for 1 h (FIG. 3E & 3F). Similar to FIGS. 3A-D, we confirmed that CD70 was expressed by IHC, and that CD27 PAMs bound to ccRCC tissue sections with greater specificity compared to scrCD27 PAMs.

### 2.4 HIF2α-CD27 PAM Treatment Reduces HIF2α mRNA Expression in Vitro

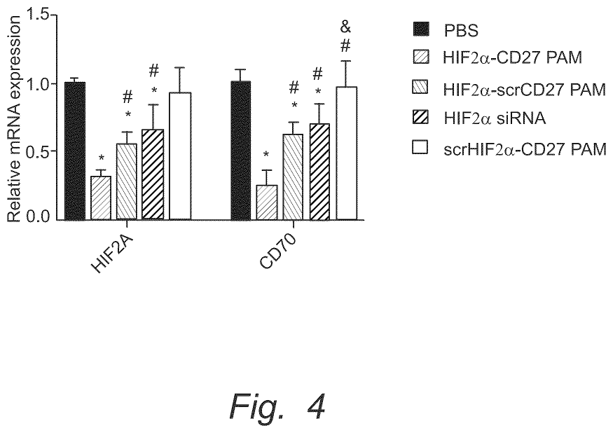

To evaluate the efficacy of siRNA delivery with CD70-targeted PAMs, patient-derived ccRCC cells were treated with HIF2α-CD27 PAMs, HIF2α-scrCD27 PAMs, scrHIF2α-CD27 PAMs, free HIF2α siRNA, or PBS for 48 h at siRNA concentrations of 500 nM. Then, HIF2A and CD70 mRNA expression were assayed through qRT-PCR (FIG. 4). Cells treated with HIF2α-CD27 PAMs were observed to have reduced HIF2A expression (31.2%±5.6%) relative to the PBS control (p<0.05). Additionally, the HIF2A knockdown mediated by HIF2α-CD27 PAMs was larger than that of non-targeting HIF2α-scrCD27 PAMs (54.8% ±9.6%, p<0.05) and free HIF2α siRNA (65.9% ±19.6%, p<0.05), suggesting that incorporation of the HIF2α siRNA into a CD70-targeted micelle increased its uptake into the ccRCC cells and thus enhanced gene silencing, which corroborates the results observed in the binding studies in FIG. 3. Similarly, HIF2α-CD27 PAM treatment also reduced CD70 mRNA expression compared to the PBS control (24.1 ±12.0), as well as the other treatment groups (p<0.05), as also reported by other groups that found CD70 expression to be correlated to HIF2α expression [55, 56].

### 2.5 HIF2α-CD27 PAMs Inhibit in Vitro Glucose Transport and Proliferation by Reducing SLC2A1 and CCND1 Expression

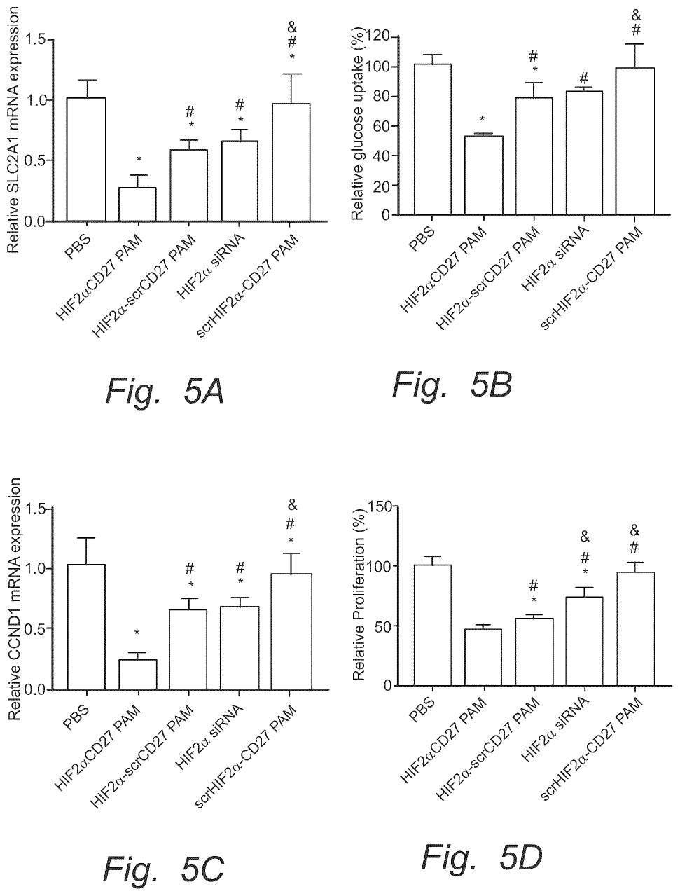

To evaluate if the gene knockdown observed in HIF2A extended to its downstream targets as well, we conducted qRT-PCR on several downstream oncogenes, starting with SLC2A1, which controls glucose uptake [57]. SLC2A1 modulates GLUT1, the transmembrane glucose transporter strongly expressed in ccRCC that contributes to increased glucose metabolism, ATP generation, and cell growth [58, 59]. To evaluate the effect of HIF2α-CD27 PAM treatment on GLUT1-mediated glucose transport, patient-derived ccRCC cells were treated with HIF2α-CD27 PAMs, HIF2α-scrCD27 PAMs, scrHIF2α-CD27 PAMs, free HIF2α siRNA, or PBS for 48 h (500 nm siRNA), and then SLC2A1 mRNA expression was assayed through qRT-PCR (FIG. 5A). HIF2α-CD27 PAMs significantly reduced SLC2A1 mRNA expression compared to HIF2α-scrCD27 PAMs, HIF2a siRNA, PBS-treated cells (p<0.05), demonstrating the potential of CD70-targeted micelles for siRNA delivery for ccRCC. To evaluate if the significant knockdown at the mRNA level impacted cell phenotype, a glucose uptake assay was performed 24 h following siRNA treatment (FIG. 5B). HIF2α-CD27 PAM treatment reduced glucose uptake by 47.8%, 32.8%, and 36.4% relative to PBS, HIF2α-scrCD27 PAMs, and free HIF2α siRNA, respectively (p<0.05).

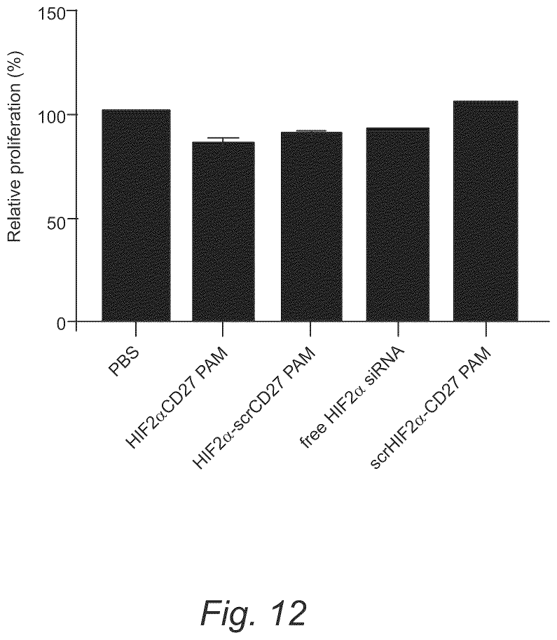

Given the ability of HIF2α-CD27 PAMs to restrict glucose metabolism in ccRCC cells, the effect of HIF2α-CD27 PAM treatment directly on ccRCC cell proliferation was examined. mRNA expression of CCND1, a downstream target of HIF2α that regulates cell cycle progression, was evaluated 48 h following HIF2α-CD27 PAM treatment (500 nM siRNA) [60, 61]. As shown in FIG. 5C, HIF2α-CD27 PAM treatment reduced CCND1 expression by 76.8% (p<0.05) and was observed to be more efficacious in knocking down CCND1 than all other groups (p<0.05). Next, the effect of HIF2α-CD27 PAM treatment on ccRCC cell proliferation was evaluated 3 d and 5 d after treatment using the MTS proliferation assay. While there was only a 15% reduction in cell proliferation in the HIF2α-CD27 PAM-treated cells after 3 d (FIG. 12), the anti-proliferative effect of micelle treatment was more pronounced 5 d after treatment, with a 54.4% reduction in cell proliferation relative to the PBS-treated control (FIG. 5D, p<0.05) and was more potent than HIF2α-scrCD27 PAMs and free HIF2α siRNA (p<0.05). These functional studies demonstrate the potential of HIF2α-CD27 PAMs to directly modulate metabolic pathways and its therapeutic ability to inhibit ccRCC cell growth and proliferation.

### 2.6 Culture Medium Collected from HIF2α-CD27 PAM-Treated ccRCC Cells Shows Anti-Angiogenic Properties

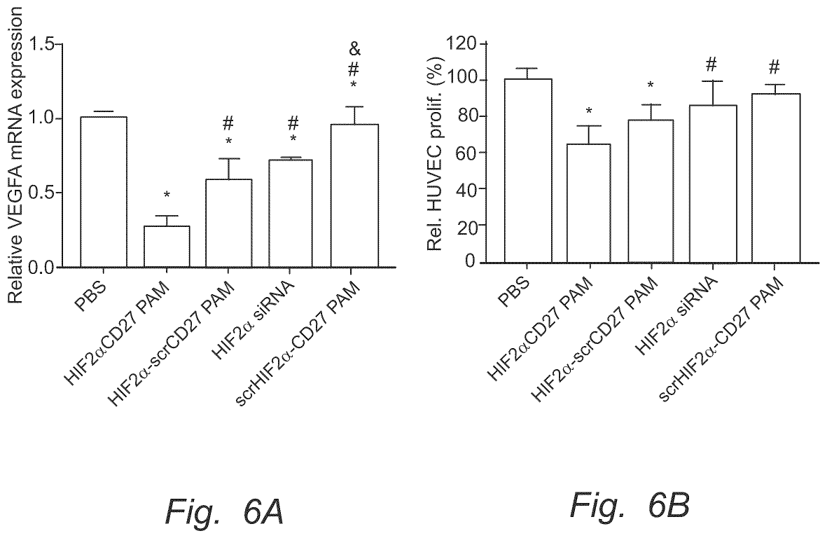

As mentioned, anti-angiogenic TKIs are often used as the clinical standard for the ccRCC treatment. Thus, to understand how HIF2α-CD27 PAMs alters the induction of angiogenesis in ccRCC tumors, we first evaluated the mRNA expression of the angiogenic VEGFA gene following PAM treatment and found that HIF2α-CD27 PAMs reduced VEGFA expression by approximately 75% compared to the PBS group (p<0.05 vs. PBS, HIF2α-scrCD27 PAMs, and free HIF2α siRNA, FIG. 6A). Then, we collected and incubated conditioned culture medium from treated patient-derived ccRCC cells with human umbilical vein endothelial cells (HUVECs) for 72 h. As shown in FIG. 6B, HUVECs cultured with conditioned medium from HIF2α-CD27 PAM-treated cells grew 40% slower than cells cultured with conditioned medium from PBS-treated cells, likely due to the reduced production and release of angiogenic growth factors in cells treated with HIF2α-CD27 PAMs (FIG. 6B) [62, 63]. These assays demonstrate the multiple, therapeutic benefits of HIF2α-CD27 PAMs that may aid to slow vascularization and growth of tumors in future in vivo studies.

### 2.7 HIF2α-CD27 PAM Treatment Reduces Patient-Derived ccRCC Cell Migration and Wound Closure

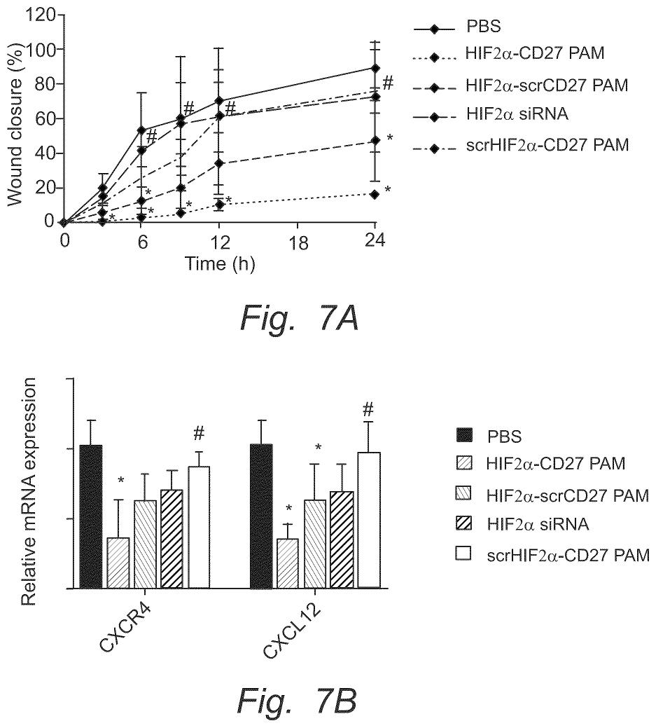

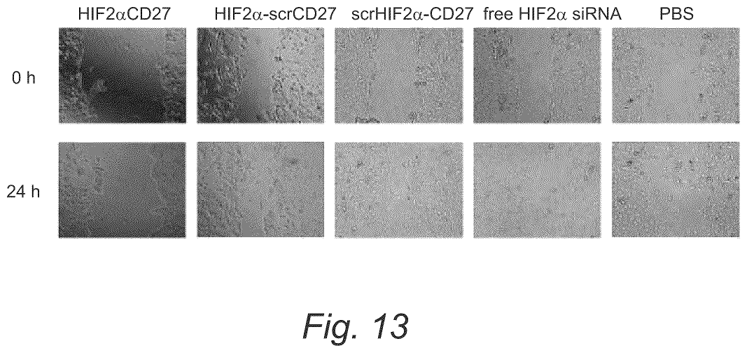

Finally, to further verify the therapeutic effects of HIF2α-CD27 PAMs, ccRCC cell migration and mobility was evaluated through wound healing assays on patient-derived ccRCC cells (FIG. 7A). Cells were treated with HIF2α-CD27 PAMs for 48 h before an artificial wound was introduced to the monolayer and cell migration was imaged up for to 24 hours. As shown in FIG. 13 and FIG. 7A, HIF2α-CD27 PAM treatment slowed wound closure by approximately 80% compared to the PBS-treated control after 24 h (p<0.05). HIF2α-scrCD27 treatment slowed wound closure by approximately 50% (p<0.05), while free HIF2α siRNA treatment did not have any significant effect on wound closure. These results are consistent with the qRT-PCR data, which shows —65% knockdown of chemotactic markers CXCR4 and CXCL12 following 48 h of HIF2α-CD27 PAM treatment, and lesser knockdown from the HIF2α-scrCD27 and free HIF2α treatments (FIG. 7B). As such, our studies collectively demonstrate that HIF2α-CD27 PAMs are capable of binding specifically to ccRCC cells and exert therapeutic effect through the modulation of HIF2α-related oncogenic genes and function.

### 3. CONCLUSION

In summary, we report the successful synthesis and characterization of peptide amphiphile micelles incorporating siRNA targeted to HIF2α and CD70-targeting peptides and its ability to exert multiple anti-tumor effects in patient-derived ccRCC cells and tumor tissues. We confirmed that incorporating CD70-targeting peptides to PAMs increased micelle binding to both patient-derived ccRCC cells in vitro and on ex vivo tissue sections. We report that HIF2α-CD27 PAMs were consistently more efficacious than both free siRNA and non-targeted PAMs in silencing gene expression and inhibiting their oncogenic functions in glucose metabolism, cell cycle progression, angiogenesis, and cell migration, highlighting the importance of both siRNA encapsulation and molecular targeting, and demonstrate the potential of HIF2α-CD27 PAMs for ccRCC-specific drug delivery. As such, future studies will evaluate the in vivo efficacy of HIF2α-CD27 PAMs using a ccRCC mouse model towards evaluating its clinical potential.

### 4. MATERIALS AND METHODS

### 4.1 Materials and Cells

Amino acids were purchased from Gyros Protein Technologies (Uppsala, Sweden) and Sigma Aldrich (St. Louis, MO, USA). PEGylated lipids were purchased from Avanti Lipids (Alabaster, AL, USA). Cy7 mono-N-hydroxysuccinimide (NHS) ester was purchased from Lumiprobe (Hunt Valley, MD, USA). DSPE-PEG2000-FITC was purchased from Creative PEGWorks (Durham, NC, USA). HIF2α and control RNA duplexes were purchased from IDT Technologies (Coralville, IA, USA). Antibodies were purchased from Thermo Fisher (Waltham, MA, USA). Cell lines were purchased from the American Type Culture Collection (ATCC, Manassas, VA, USA). Cell culture reagents were purchased from Gibco (Waltham, MA, USA) and Sigma Aldrich. Human clear cell renal cell carcinoma (ccRCC) tissue samples were generously donated from UCLA urology.

### 4.2 Synthesis of HIF2α-CD27 Peptide Amphiphile Micelles

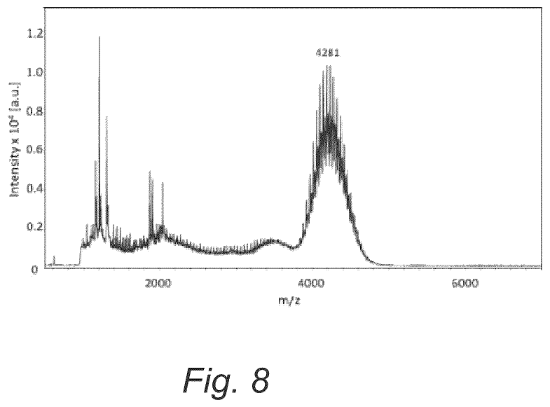

CD27 (CRKAAQCDPCIPG) and scrambled CD27 (CQGPRACKADPIC) peptides were synthesized on a Rink amide resin using Fmoc-mediated solid phase peptide synthesis with an automated peptide synthesizer (PS3, Gyros Protein Technologies) and N-capped with an acetyl group. Peptide deprotection and cleavage from the resin was performed with two 2 h incubations with a 94:2.5:2.5:1 vol% mixture of trifluoroacetic acid (TFA):water:ethanedithiol:triisopropylsilane (TIS). To control conjugation to DSPE-PEG2000-maleimide, peptides were synthesized with TFA-stable acetamidomethyl (Acm) groups protecting the side chains of the non-terminal cysteines, which were deprotected later with 10 eq. mercury acetate. Peptides were purified using reverse-phase high performance liquid chromatography (RP-HPLC, Prominence, Shimadzu, Columbia, MD, USA) on a Luna C18 column (Phenomenex, Torrance, CA, USA) at 55° C. using HPLC-grade water and acetonitrile (Fisher Scientific, Hampton, NH, USA) supplemented with 0.1% formic acid. Peptide purity was characterized using matrix-assisted laser desorption/ionization time-of-flight mass spectrometry (MALDI-TOF-MS, Bruker, MA, USA). The expected m/z is 1403 (FIG. 8).

Pure peptides were then mixed with a 10% molar excess of DSPE-PEG2000-maleimide in water before adjustment of the pH to 7.2. The reaction was nitrogen purged and allowed to stir for 3 d before purification through RP-HPLC using a Luna C4 column (Phenomenex). Acm groups were removed using 10 eq. of mercury acetate, and the pure peptide amphiphiles were desalted through RP-HPLC and characterized through MALDI-TOF-MS. The expected m/z is 4343 m/z (FIG. 9). Fluorescent DSPE-PEG2000-cy7 amphiphiles were synthesized by dissolving DSPE-PEG2000-amine in a 0.1 M sodium bicarbonate buffer, and then mixing it with 3-fold molar excess of cy7 NHS ester solubilized in dimethyl formamide (DMF, 10% of reaction volume). The reaction was allowed to shake overnight before purification using a Luna C4 column and characterization via MALDI-TOF-MS. HIF2α siRNA (sense: 5′ -CUUGCAGUUUUACUAAAACACUGAA-3′, antisense: 5′ -UUCAGUGUUUUAGUAAAACUGCAAGGG-3′) thiolated on the 5′ end of the sense strand was conjugated to DSPE-PEG2000-maleimide through a thioether bond by mixing the siRNA and lipid in nuclease-free water adjusted to pH 7.2 overnight. Conjugation of DSPE-PEG2000-HIF2α siRNA amphiphiles was confirmed through the gel electrophoresis assays described in 4.5.

Micelles were self-assembled from peptide and siRNA amphiphiles through thin-film hydration. Peptide amphiphiles were dissolved in methanol, and a nitrogen stream was used to evaporate the methanol, leaving a thin lipid film that was then hydrated with nuclease-free water or PBS containing the siRNA amphiphiles, gently sonicated and vortexed, and heated to 40° C. for 30 min. Unless otherwise stated, siRNA and fluorescent amphiphiles were incorporated into micelles at 1 mol % and 10 mol %, respectively.

### 4.3 Dynamic Light Scattering (DLS) and Zeta Potential

Micelles were suspended at 50 μM in 1 mM NaCl in a folded capillary zeta cell for size, polydispersity, and zeta potential measurements using a Zetasizer Ultra instrument (Malvern Panalytical, Malvern, UK) at room temperature (n=3).

### 4.4 Transmission Electron Microscopy (TEM)

Micelles were imaged using a FEI Talos F200C microscope (Thermo Fisher). 5 uL of a 25 μM micelle solution in water was placed directly onto a 400 mesh Carbon Type-B TEM grid (Ted Pella, Redding, CA, USA), washed with water, and then stained with 2% uranyl acetate before a final wash. Grids were left overnight in the dark prior to imaging.

### 4.5 Gel Electrophoresis Assay

HIF2α-CD27 (500 ng siRNA, 13.3 1.tg CD27) PAMs or free HIF2α siRNA (500 ng siRNA) was loaded into the wells of a 2% (w/v) agarose gel containing 0.5 μg/mL ethidium bromide and RNA migration was observed using a ChemiDoc XRS+ imaging system (Bio-Rad, Hercules, CA, USA) after the application of 50V for 90 min. For RNase treatment, samples were incubated for 1 h with 50 μg/mL RNase A (Thermo Fisher) in 5% fetal bovine serum (FBS) prior to gel electrophoresis.

### 4.6 Characterization of siRNA Release

300 μM HIF2α-CD27 micelles were incubated with 10 mM glutathione (GSH) for up to 24 h and then gel electrophoresis was performed under the conditions detailed in 4.5. Relative band intensity was quantified using ImageJ and used to calculate % siRNA release.

### 4.7 Isolation and Culture of Patient-Derived ccRCC Cells

Patient tumor samples were a generous gift from the UCLA Urology department. Tumors were transported on ice before dicing finely using a pair of scalpels. Afterwards, samples were digested in low-serum culture media containing 2 mg/mL Type-II collagenase and 2% penicillin-streptomycin overnight at 37° C., passed sequentially through 70 um and 40 μm filters, and then washed twice in fresh RPMI before seeding onto collagen-coated tissue culture plates or flasks. Cells were cultured in RPMI supplemented with 10-20% FBS and 1% penicillin-streptomycin.

### 4.8 In Vitro Micelle Binding to Patient-Derived ccRCC Cells

Approximately 200,000 patient-derived ccRCC cells or HK-2 cells were seeded onto glass coverslips placed in the wells of a 6-well plate and allowed to adhere overnight. 50 μM FITC-labeled CD27 or scrambled CD27 micelles were incubated with the cells for 30 min at 37° C. and then fixed with fresh 4% paraformaldehyde (PFA) and blocked with 0.3 M glycine and 5% normal goat serum in PBS-Tween-20 (PBST). After blocking, cells were incubated with anti-CD70 primary antibodies (1:200) overnight at 4° C. and then AF594 secondary antibodies (1:500) for 1 h at room temperature. Cells were then counterstained with 2 μg/mL DAPI and mounted to Superfrost Plus microscope slides (Fisher) using Vectamount aqueous mounting media (Vector Laboratories, Burlingame, CA, USA) and sealing with clear nail polish. Microscope slides were allowed to set overnight in the dark at room temperature before imaging on an LSM 880 confocal microscope. In addition to microscopy, 10,000 cells were also seeded into black 96-well plates, incubated with 5011M FITC-labelled micelles for 30 min, washed, and then fluorescence was measured using a Synergy H4 Hybrid microplate reader (Agilent, Santa Clara, CA, USA, n=6). To block cell surface CD70, cells were incubated with anti-CD70 (1:20) for 30 min at 37° C. prior to micelle incubation.

### 4.9 Immunohistochemical (IHC) Staining and PAM Binding to ccRCC Patient Tissue Sections

ccRCC patient tumor samples were formalin-fixed and paraffin-embedded (FFPE) and sectioned at 101.tm. Sections were then stained with hematoxylin and eosin or prepared for immunofluorescent (IF) antibody staining. Briefly, for IF staining, tissue sections were heated to 100 ° C. in pH 6 citrate buffer for 15 min, blocked with 5% normal goat serum in TB S-Tween-20 (TBST) for 30 min, incubated with anti-CCR2 (1:200), anti-LRP2 (1:200), or anti-CD70 (1:200) primary antibodies at 4° C. overnight, washed with TBST and then incubated for 1 h with Alexa Fluor 594-conjugated secondary antibodies (1:500) at room temperature. Following antibody staining, tissue sections were washed and then incubated with 10 μM CD27 micelles for 1 h before washing and counterstaining with 21.tg/mL DAPI. Coverslips were mounted using Prolong Gold mounting medium (Thermo Fisher) and allowed to cure overnight before imaging using a Zeiss LSM 880 confocal microscope (Zeiss, Oberkochen, DE).

### 4.10 In vitro Transfection and mRNA Expression of Patient-Derived ccRCC Cells

100 μL micelles or free siRNA was added to the wells of a 12-well plate at a concentration of 5 μM RNA, and then 900 uL of a solution containing approximately 100,000 cells in 5% FBS/RPMI was added (n=5). Cells were incubated for 48 h at 37° C., after which the cells were lysed and RNA was isolated using the mRNeasy Kit from Qiagen (Hilden, DE). cDNA was synthesized using the RT2 First Strand Kit according to manufacturer instructions (Qiagen). Expression of HIF2A, CD70, SLC2A1, CCND1, VEGFA, CXCR4, CXCL12, and GAPDH was evaluated through real-time PCR with primer assays and the RT2 SYBR Green qPCR Mastermix (Qiagen) using a CFX384 Touch Real-Time PCR Detection System (Bio-Rad Laboratories), according to manufacturer instructions. Fold-change in mRNA expression was calculated using the delta-delta Ct method.

### 4.11 MTS Assay

In vitro micelle biocompatibility was evaluated by incubating micelles or siRNA with patient-derived RCC or HK-2 cells for 48 h in 5% FBS/RPMI before adding MTS reagent (10% v/v, Abcam, Cambridge, UK) and incubating for 1 h. Afterwards, absorbance at 490 nm was quantified using a microtiter plate reader. Cell % viability was determined following blank subtraction by normalizing the treatment values to PBS-treated control wells. The effect of siRNA transfection on cell proliferation was determined by transfecting cells according to 4.10, replacing the transfection solution with fresh complete culture media, and then incubating for an additional 48-120 h before adding the MTS reagent for subsequent absorbance measurements (n=5).

### 4.12 Glucose Uptake Assay

5,000 cells were plated in the wells of a 96-well plate (n=4) and treated with HIF2α-CD27, HIF2α-scrCD27, scrHIF2α-CD27, HIF2α, or PBS at an siRNA concentration of 500 nM for 48 h at 37° C. Afterwards, the cell culture media was refreshed, and the cells were allowed to grow unperturbed for 24 h before evaluation of glucose uptake using the Glucose Uptake-Glo assay (Promega, Madison, WI, USA) according to manufacturer instructions. Glucose uptake was evaluated via luminescence measurements from a Synergy H4 Hybrid Microplate Reader.

### 4.13 Collection of ccRCC-Conditioned Cell Culture Medium and in Vitro Culture and Growth of Endothelial Cells

ccRCC cells were transfected as detailed in 4.10, then cell culture medium was refreshed. After 24 h, conditioned culture medium was collected and stored at 4° C. 5,000 HUVECs were seeded into the wells of a 96-well plate and allowed to adhere and grow over 48 h. Then, conditioned culture medium was diluted 1:1 in a 4:1 mixture of serum-free RPMI and endothelial cell growth medium. This mixture was added to the HUVECs, and the cells were allowed to grow for 72 h before evaluation of cell proliferation via MTS assay (n=5).

### 4.14 Wound Healing Assay

50,000 cells were seeded into the wells of a 48-well plate (n=4) and cultured for 5 d to attain a confluent monolayer. Then, cells were treated with HIF2α-CD27, HIF2α-scrCD27, scrHIF2α-CD27, HIF2α, or PBS according to 4.10. After treatment, the media in the wells was replaced with fresh culture media and a 200 uL pipette tip was used to create a single scratch on the surface of the wells, and brightfield images of the cells were taken with a Leica DMi8 microscope (Leica, Wetzlar, DE) 0, 3, 6, 9, 12, and 24 h after scratching. A scalpel blade was used to mark the wells to ensure microscope images were taken in the same field-of-view for each timepoint. Wound closure was measured as the area of the scratch using ImageJ.

### 4.15 Statistics

Data are expressed as Mean ±SD. Statistical analysis between two groups was performed using a Student's t-test. Comparisons between three or more groups was performed through analysis of variance (ANOVA) followed by post-hoc Dunnett's test for multiple comparisons. A p-value <0.05 was considered to be statistically significant.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

### REFERENCES

1. Padala, S. A., et al., Epidemiology of Renal Cell Carcinoma. World J Oncol, 2020. 11(3): p. 79-87.
2. Wierzbicki, P. M., et al., Prognostic significance of VHL, HIF1A, HIF2A, VEGFA and p53 expression in patients with clearcell renal cell carcinoma treated with sunitinib as firstline treatment. Int J Oncol, 2019. 55(2): p. 371-390.
3. Dagher, J., et al., Clear cell renal cell carcinoma: a comparative study of histological and chromosomal characteristics between primary tumors and their corresponding metastases. Virchows Arch, 2017. 471(1): p. 107-115.
4. Serzan, M.T . and M. B. Atkins, Current and emerging therapies for first line treatment of metastatic clear cell renal cell carcinoma. J Cancer Metastasis Treat, 2021. 7.
5. Motzer, R. J., et al., Avelumab plus Axitinib versus Sunitinib for Advanced Renal-Cell Carcinoma. N Engl J Med, 2019. 380(12): p. 1103-1115.
6. Motzer, R., et al., Lenvatinib plus Pembrolizumab or Everolimus for Advanced Renal Cell Carcinoma. N Engl J Med, 2021. 384(14): p. 1289-1300.
7. Razafinjatovo, C., et al., Characterization of VHL missense mutations in sporadic clear cell renal cell carcinoma: hotspots, affected binding domains, functional impact on pVHL and therapeutic relevance. BMC Cancer, 2016. 16: p. 638.
8. Nickerson, M. L., et al., Improved identification of von Hippel-Lindau gene alterations in clear cell renal tumors. Clin Cancer Res, 2008. 14(15): p. 4726-34.
9. Young, A. C., et al., Analysis of VHL Gene Alterations and their Relationship to Clinical Parameters in Sporadic Conventional Renal Cell Carcinoma. Clin Cancer Res, 2009. 15(24): p. 7582-7592.
10. Baldewijns, M. M., et al., VHL and HIF signalling in renal cell carcinogenesis. J Pathol, 2010. 221(2): p. 125-38.
11. Ruf, M., H. Moch, and P. Schraml, PD-Ll expression is regulated by hypoxia inducible factor in clear cell renal cell carcinoma. Int J Cancer, 2016. 139(2): p. 396-403.
12. Martinez-Saez, O., et al., Targeting HIF-2 alpha in clear cell renal cell carcinoma: A promising therapeutic strategy. Crit Rev Oncol Hematol, 2017. 111: p. 117-123.
13. Arnaiz, E., et al., Differential effects of HIF2alpha antagonist and HIF2alpha silencing in renal cancer and sensitivity to repurposed drugs. BMC Cancer, 2021. 21(1): p. 896.
14. Micucci, C., et al., HIF2alpha is involved in the expansion of CXCR4-positive cancer stem-like cells in renal cell carcinoma. Br J Cancer, 2015. 113(8): p. 1178-85.
15. Jonasch, E., et al., Belzutifan for Renal Cell Carcinoma in von Hippel-Lindau Disease. N Engl J Med, 2021. 385(22): p. 2036-2046.
16. McCabe, E. M., S. Lee, and T. P. Rasmussen, Belzutifan (Welireg) for von Hippel Lindau disease. Trends Pharmacol Sci, 2022.
17. Wong, S. C., et al., HIF2alpha-Targeted RNAi Therapeutic Inhibits Clear Cell Renal Cell Carcinoma. Mol Cancer Ther, 2018. 17(1): p. 140-149.
18. Rankin, E. B., et al., Hypoxia-inducible factor-2 (HIF-2) regulates hepatic erythropoietin in vivo. J Clin Invest, 2007. 117(4): p. 1068-77.
19. Huang, Y., et al., Improving kidney targeting: The influence of nanoparticle physicochemical properties on kidney interactions. J Control Release, 2021. 334: p. 127-137.
20. Wang, J., et al., Oral delivery of metformin by chitosan nanoparticles for polycystic kidney disease. J Control Release, 2021. 329: p. 1198-1209.
21. Wang, J., N. Tripathy, and E. J. Chung, Targeting and therapeutic peptide-based strategies for polycystic kidney disease. Adv Drug Deliv Rev, 2020. 161-162: p. 176-189.
22. Trac, N. T. and E. J. Chung, Peptide-based targeting of immunosuppressive cells in cancer. Bioact Mater, 2020. 5(1): p. 92-101.
23. Trac, N. and E. J. Chung, Overcoming physiological barriers by nanoparticles for intravenous drug delivery to the lymph nodes. Exp Biol Med (Maywood), 2021. 246(22): p. 2358-2371.
24. Wang, G., et al., Kidney-targeted rhein-loaded liponanoparticles for diabetic nephropathy therapy via size control and enhancement of renal cellular uptake. Theranostics, 2019. 9(21): p. 6191-6208.
25. Kamaly, N., et al., Nanomedicines for renal disease: current status and future applications. Nat Rev Nephrol, 2016. 12(12): p. 738-753.
26. Wang, J., et al., Design and in vivo characterization of kidney-targeting multimodal micelles for renal drug delivery. Nano Research, 2018. 11(10): p. 5584-5595.
27. Tripathy, N., et al., Transdermal Delivery of Kidney-Targeting Nanoparticles Using Dissolvable Microneedles. Cell Mol Bioeng, 2020. 13(5): p. 475-486.
28. Pal, S. K., et al., A phase 1 trial of SGN-CD70A in patients with CD70-positive, metastatic renal cell carcinoma. Cancer, 2019. 125(7): p. 1124-1132.
29. Teplyakov, A., et al., Crystal structure of CD27 in complex with a neutralizing noncompeting antibody. Acta Crystallogr F Struct Biol Commun, 2017. 73(Pt 5): p. 294-299.
30. Huang, Y., et al., The effect of size, charge, and peptide ligand length on kidney targeting by small, organic nanoparticles. Bioeng Transl Med, 2020. 5(3): p. e10173.
31. Poon, C., et al., Protein Mimetic and Anticancer Properties of Monocyte-Targeting Peptide Amphiphile Micelles. ACS Biomater Sci Eng, 2017. 3(12): p. 3273-3282.
32. Trac, N., et al., CCR2-targeted micelles for anti-cancer peptide delivery and immune stimulation. J Control Release, 2021. 329: p. 614-623.
33. Patel, N., et al., Therapeutic Response of miR-145 Micelles on Patient-Derived Vascular Smooth Muscle Cells. Front Digit Health, 2022. 4: p. 836579.
34. Sato, Y., et al., Neutralization of negative charges of siRNA results in improved safety and efficient gene silencing activity of lipid nanoparticles loaded with high levels of siRNA. J Control Release, 2018. 284: p. 179-187.
35. Kasai, H., et al., Efficient siRNA delivery and gene silencing using a lipopolypeptide hybrid vector mediated by a caveolae-mediated and temperature-dependent endocytic pathway. J Nanobiotechnology, 2019. 17(1): p. 11.
36. Chin, D. D., et al., miR-145 micelles mitigate atherosclerosis by modulating vascular smooth muscle cell phenotype. Biomaterials, 2021. 273: p. 120810.
37. Jeong, E. J., et al., In Vitro Cellular Uptake and Transfection of Oligoarginine-Conjugated Glycol Chitosan/siRNA Nanoparticles. Polymers (Basel), 2021. 13(23).
38. Houseley, J. and D. Tollervey, The many pathways of RNA degradation. Cell, 2009. 136(4): p. 763-76.
39. Aranda, P. S., D. M. LaJoie, and C. L. Jorcyk, Bleach gel: a simple agarose gel for analyzing RNA quality. Electrophoresis, 2012. 33(2): p. 366-9.
40. Lushchak, V. I., Glutathione homeostasis and functions: potential targets for medical interventions. J Amino Acids, 2012. 2012: p. 736837.
41. Forman, H. J., H. Zhang, and A. Rinna, Glutathione: overview of its protective roles, measurement, and biosynthesis. Mol Aspects Med, 2009. 30(1-2): p. 1-12.
42. Lv, H., et al., Unraveling the Potential Role of Glutathione in Multiple Forms of Cell Death in Cancer Therapy. Oxid Med Cell Longev, 2019. 2019: p. 3150145.
43. Xiao, Y. and D. Meierhofer, Glutathione Metabolism in Renal Cell Carcinoma Progression and Implications for Therapies. Int J Mol Sci, 2019. 20(15).
44. Kurozumi, R. and S. Kojima, Increase of intracellular glutathione by low-level NO mediated by transcription factor NF-kappaB in RAW 264.7 cells. Biochim Biophys Acta, 2005. 1744(1): p. 58-67.
45. Deponte, M., Glutathione catalysis and the reaction mechanisms of glutathione-dependent enzymes. Biochim Biophys Acta, 2013. 1830(5): p. 3217-66.
46. Melendez-Rodriguez, F., et al., Hypoxia-Inducible Factor 2-Dependent Pathways Driving Von Hippel-Lindau-Deficient Renal Cancer. Front Oncol, 2018. 8: p. 214.
47. Chen, W., et al., Targeting renal cell carcinoma with a HIF-2 antagonist. Nature, 2016. 539(7627): p. 112-117.
48. Adam, P. J., et al., CD70 (TNFSF7) is expressed at high prevalence in renal cell carcinomas and is rapidly internalised on antibody binding. Br J Cancer, 2006. 95(3): p. 298-306.
49. Raval, R. R., et al., Contrasting properties of hypoxia-inducible factor 1 (HIF-1) and HIF-2 in von Hippel-Lindau-associated renal cell carcinoma. Mol Cell Biol, 2005. 25(13): p. 5675-86.
50. Shinojima, T., et al., Renal cancer cells lacking hypoxia inducible factor (HIF)-lalpha expression maintain vascular endothelial growth factor expression through HIF-2alpha. Carcinogenesis, 2007. 28(3): p. 529-36.
51. Choi, W. S. W., J. Boland, and J. Lin, Hypoxia-Inducible Factor-2alpha as a Novel Target in Renal Cell Carcinoma. J Kidney Cancer VHL, 2021. 8(2): p. 1-7.
52. Delahunt, B., Advances and controversies in grading and staging of renal cell carcinoma. Mod Pathol, 2009. 22 Suppl 2: p. S24-36.
53. Warren, A. Y. and D. Harrison, WHO/ISUP classification, grading and pathological staging of renal cell carcinoma: standards and controversies. World J Urol, 2018. 36(12): p. 1913-1926.
54. Samaratunga, H., T. Gianduzzo, and B. Delahunt, The ISUP system of staging, grading and classification of renal cell neoplasia. J Kidney Cancer VHL, 2014. 1(3): p. 26-39.
55. Ruf, M., et al., pVHL/HIF-regulated CD70 expression is associated with infiltration of CD27+lymphocytes and increased serum levels of soluble CD27 in clear cell renal cell carcinoma. Clin Cancer Res, 2015. 21(4): p. 889-98.
56. Kitajima, S., et al., Hypoxia-inducible factor-2 alpha up-regulates CD70 under hypoxia and enhances anchorage-independent growth and aggressiveness in cancer cells. Oncotarget, 2018. 9(27): p. 19123-19135.
57. Pragallapati, S. and R. Manyam, Glucose transporter 1 in health and disease. J Oral Maxillofac Pathol, 2019. 23(3): p. 443-449.
58. Chan, D. A., et al., Targeting GLUT1 and the Warburg effect in renal cell carcinoma by chemical synthetic lethality. Sci Transl Med, 2011. 3(94): p. 94ra70.
59. Adekola, K., S. T. Rosen, and M. Shanmugam, Glucose transporters in cancer metabolism. Curr Opin Oncol, 2012. 24(6): p. 650-4.
60. Fu, M., et al., Minireview: Cyclin D1 : normal and abnormal functions. Endocrinology, 2004. 145(12): p. 5439-47.
61. Al ao, J. P., The regulation of cyclin D1 degradation: roles in cancer development and the potential for therapeutic invention. Mol Cancer, 2007. 6: p. 24.
62. Alhawarat, F. M., et al., The effect of cycling hypoxia on MCF-7 cancer stem cells and the impact of their microenvironment on angiogenesis using human umbilical vein endothelial cells (HUVECs) as a model. PeerJ, 2019. 7: p. e5990.
63. Jin, S., et al., Conditioned medium derived from FGF-2-modified GMSCs enhances migration and angiogenesis of human umbilical vein endothelial cells. Stem Cell Res Ther, 2020. 11(1): p. 68.

What is claimed is:

1. A drug delivery system comprising:
a plurality of nanoparticles wherein each nanoparticle includes targeting peptides and/or targeting antibodies and/or targeting aptamers that target a renal carcinoma marker conjugated thereto and a therapeutic for renal carcinoma conjugated thereto, the therapeutic for renal carcinoma selected from the group consisting of siRNAs, microRNAs, mRNA, DNA, nucleic acids, small molecule drugs, and combinations thereof.

2. The drug delivery system of claim 1, wherein the targeting peptides includes CD70-targeting peptides.

3. The drug delivery system of claim 1, wherein the therapeutic for renal carcinoma includes anti-HIF2α siRNAs.

4. The drug delivery system of claim 2 wherein the CD70-targeting peptides conjugated includes a fragment of CD27 (CRKAAQCDPCIPG) having 5 to 12 amino acids or peptide thereof with 1 to 7 conservative substitutions or additions.

5. The drug delivery system of claim 2, wherein the plurality of nanoparticles includes nanoparticles selected from the group consisting of micelles, liposomes, and combinations thereof

6. The drug delivery system of claim 2, wherein the CD70-targeting peptides is CD27 (CRKAAQCDPCIPG) (SEQ ID NO: 1) or a peptide thereof with 1 to 7 conservative substitutions or additions.

7. The drug delivery system of claim 1 wherein anti-HIF2α siRNAs include a polynucleotide having sequence 5′ -CUUGCAGUUUUACUAAAACACUGAA-3′ (SEQ ID NO: 2) and/or an antisense sequence: 5′-UUCAGUGUUUUAGUAAAACUGCAAGGG-3′ (SEQ ID NO: 3) or sequences having a sequence identity greater than 80 percent identity thereof

8. The drug delivery system of claim 1 wherein anti-HIF2α siRNAs include a fragment of a polynucleotide having sequence 5′-CUUGCAGUUUUACUAAAACACUGAA-3′ (SEQ ID NO:

2. and/or an antisense sequence: 5′-UUCAGUGUUUUAGUAAAACUGCAAGGG-3′ 3′ (SEQ ID NO: 3) having 5 to 30 nucleotides or sequences having a sequence identity greater than 80 percent identity thereof

9. The drug delivery system of claim 1, wherein CD70-targeting peptides and anti-HIF2a siRNAs are independently connected to the nanoparticles by reaction with a functional group.

10. The drug delivery system of claim 9, wherein the functional group that can be used for linking includes amines, carboxylic acids, NHS esters, acid anhydrides, or unsaturated imides.

11. The drug delivery system of claim 1, wherein CD70-targeting peptides and anti-HIF2a siRNAs are independently absorbed electrostatically to the nanoparticles.

12. The drug delivery system of claim 1 wherein the plurality of nanoparticles includes a plurality of micelles.

13. The drug delivery system of claim 12 wherein each micelle includes a plurality of targeting peptide-conjugated amphiphiles, a plurality of therapeutic agent-conjugated amphiphiles, and an optional plurality of non-targeted amphiphiles.

14. The drug delivery system of claim 13, wherein the plurality of targeting peptide-conjugated amphiphiles includes amphiphiles having a first phospholipid conjugated to CD70-targeting peptides with a first linking group, the plurality of therapeutic agent-conjugated amphiphiles includes amphiphiles having a second phospholipid conjugated to anti-HIF2α siRNAs with a second linking group, and the optional plurality of non-targeted amphiphiles includes amphiphiles having a third phospholipid conjugated to a capping moiety with a third linking group.

15. The drug delivery system of claim 14, wherein the first linking group, the second linking group, and the third linking group are each independently a polyethylene glycol having a weight average molecular weight from about 500 to 10000 Daltons.

16. The drug delivery system of claim 14, wherein the first phospholipid, the second phospholipid, and the third phospholipid are each independently selected from the group consisting of phosphatidic acids, phosphatidyl inositols, phosphatidyl cholines, phosphatidyl ethanolamines, phosphatidyl serines, phosphatidyl glycerols, and any combinations thereof.

17. The drug delivery system of claim 14 wherein the first phospholipid, the second phospholipid, and the third phospholipid are each independently selected from the group consisting of phosphatidylglycerol, lecithin, sphingomyelin, phosphatidylserine, phosphatidic acid, N-(2,3-di(9-(Z)-octadecenyloxy))-prop-1-yl-N,N,N-trimethylammonium chloride, phosphatidylethanolamine, lysolecithin, lysophosphatidylethanolamine, phosphatidylinositol, cephalin, cardiolipin, cerebrosides, dicetylphosphate, dioleoylphosphatidylcholine, dipalmitoylphosphatidylcholine, dipalmitoylphosphatidylglycerol, dioleoylphosphatidylglycerol, palmitoyl-oleoyl-phosphatidylcholine, di-stearoyl-phosphatidylcholine, stearoyl-palmitoyl-phosphatidylcholine, di-palmitoyl-phosphatidylethanolamine, di-stearoyl-phosphatidylethanolamine, di -myrstoyl-phosphatidylserine, di-oleyl-phosphatidylcholine, dimyristoyl phosphatidyl choline (DMPC), dioleoylphosphatidylethanolamine, palmitoyloleoylphosphatidylcholine, di stearoylphosphatidylcholine, dioleoylphosphatidylcholine, dipalmitoylphosphatidylcholine, dioleoylphosphatidylglycerol, dipalmitoylphosphatidylglycerol, -phosphatidylethanolamine, dioleoyl-phosphatidylethanolamine 4-(N-maleimidomethyl)-cyclohexane-1-carboxylate (DOPE-mal), 1-stearoyl-2-oleoyl phosphatidylcholine, 1,2-distearoyl-sn-glycerol-3-phosphoethanolamine, and combinations thereof.

18. The drug delivery system of claim 13 further comprising a pharmaceutically acceptable carrier.

19. The drug delivery system of claim 12, wherein at least a subset of the micelles further includes a drug incorporated into a core of each micelle or conjugated to a hydrophobic tail of a therapeutic agent-conjugated amphiphile.

20. The drug delivery system of claim 12 wherein at least a subset of the micelles include a component selected from the group consisting of small molecule therapeutics, chemotherapeutic agents, monoclonal antibodies, therapeutic cytokines, and tyrosine kinase inhibitors which are currently used for treating renal carcinoma incorporated therein.

21. A method for treating renal cancer in a subject, the method comprising:
identifying a subject having renal cancer; and
administering a therapeutically effective amount of a drug delivery system comprising:
a plurality of nanoparticles wherein each nanoparticle includes targeting peptides and/or targeting antibodies and/or targeting aptamers that target a renal carcinoma marker conjugated thereto and a therapeutic for renal carcinoma conjugated thereto, the therapeutic for renal carcinoma selected from the group consisting of siRNAs, microRNAs, mRNA, DNA, nucleic acids, small molecule drugs, and combinations thereof

22. The method of claim 21, wherein a small molecule therapeutic and/or chemotherapeutic agents, and/or tyrosine kinase inhibitors and/or monoclonal antibodies and/or therapeutic cytokines which are currently used for treating renal carcinoma is incorporated into the nanoparticles.

23. The method of claim 21, wherein the drug delivery system is administered intravenously, subcutaneously, orally, by intraperitoneal injection, and transdermally.

24. The method of claim 21, wherein the renal cancer is clear cell renal carcinoma.
